(19) United States
(12) Patent Application Publication
(10) Pub. No.: US 2025/0064566 A1
(43) Pub. Date: Feb. 27, 2025

(54) MAGNETIC FLOSS

(71) Applicant: LEISHA ARMIJO MARTIN, Corpus Christi, TX (US)

(72) Inventor: LEISHA ARMIJO MARTIN, Corpus Christi, TX (US)

(21) Appl. No.: 18/731,324

(22) Filed: Jun. 2, 2024

Related U.S. Application Data
(60) Provisional application No. 63/470,516, filed on Jun. 2, 2023.

(51) Int. Cl. A61C 15/00; A61C 19/06; A61K 8/19; A61K 8/22; A61K 8/38; A61N 5/06; A61Q 11/00

(52) U.S. Cl. CPC A61C 15/00; A61C 19/06; A61K 8/19; A61K 8/22; A61K 8/38; A61N 5/0603; A61N 5/0613; A61Q 11/00; A61K 2800/413; A61K 2800/47; A61N 2005/0606; A61N 2005/0645; A61N 2005/0651; A61N 2005/0659; A61N 2005/0663

(57) ABSTRACT

An apparatus and methods or delivering and controlling magnetic abrasive particles to clean between teeth and at and just below the gum line by removing debris. The apparatus can include a horseshoe-shaped optically clear mouth tray (for upper and lower teeth) appropriately sized to fit the teeth (ridge) and over the gum line of a patient. Magnetic nano or microparticles can be manipulated within a ferrofluid mixture by a magnetic field. The ferrofluid, teeth and gumline can be illuminated by a light source from or through the mouth tray.

### INVENTION PRIORITY

The present invention is a continuation of Provisional Application Ser. No. 63/470,516 entitled “Magnetic Floss”, which was filed on Jun. 2, 2023, and is hereby incorporated by reference.

### FIELD OF THE INVENTION

The present invention generally relates to method and system that enhance oral healthcare. The present invention also relates to the fields of oral hygiene, dentistry, orthodontics and cosmetic dentistry. More particularly, the present invention relates to an apparatus for a type of “string-free” flossing for removing debris from the surfaces of the teeth, between the teeth, and below the gum line. The major function is that of an abrasive. The invention can encompass a type of flexible, polymeric mouth tray that can hold the applied magnetic particle ferrofluid at the area of interest and can manipulate the ferrofluid with an external magnetic field, applied to ferrofluid within a U-shaped mouth trough.

### BACKGROUND OF THE INVENTION

Numerous devices and techniques are available to improve oral hygiene and contribute to aesthetics of the teeth and gums. These include peroxide-based whitening products, manual and electric toothbrushes, water flossers, string floss, dental abrasives, toothpastes and mouthwashes. All these products are limited by their ability to physically contact and mechanically or chemically alter the surfaces they encounter.

Certain types of bacteria are ultimately to blame for cavities and bleeding gums (gum disease) in addition to tooth decay and enamel deterioration. Inadequate cleaning is a common cause of bacterial or plaque buildup on the teeth and gums.

Bacterial biofilms can form in the oral cavity, especially on inert surfaces, such as orthodontic wires, bridges, dental fillings, etc. Bacterial biofilms cause inflammation and immune dysregulation and lead to gingivitis, periodontitis, bone and tooth loss. The present invention consists of an apparatus to treat oral bacterial, fungal and viral pathogens, including those located in places where typical mechanical devices, brushes and floss are unable to penetrate.

It is known that magnetic nano and microscale particles can be engineered with antimicrobial properties, by surface interactions, release of metal ions, reactive oxygen species, and/or antibacterial drug conjugation. The present inventor has determined that the combination of magnetic nanoparticles with an external static, switched or oscillating magnetic field provides an enhanced effect in bacterial biofilm eradication. Nanoparticles are now being used for various human therapies. Examples of nanoparticle use can are shown in US Patent publication: U.S. Pat. Nos. 9,186,317B2, 9,153,637B2 by the present inventor, and US Application 2021/0393815A1 of which the present inventor is a co-inventor. These references are incorporated by reference herein in their entirety.

What is still needed are additional human therapies that incorporate nanoparticles in a manner that can benefit the field of oral healthcare. What is needed are new methods, systems and apparatuses that help prevent initial or additional damage to the teeth and gums by effectively cleaning the teeth and gums.

### SUMMARY OF THE INVENTION

It is a feature of the present embodiments to provide systems and methods to support oral healthcare. For example, embodiments can improve dental hygiene for people suffering from periodontal disease, gingivitis, and complication of orthodontic devices, wires, crows and bridges. A flossing system that can be remotely controlled, and in which the abrasive particles can reach previously unreachable areas of the mouth, are of interest. Such a system would be especially valuable to people with orthodontic wires, devices, bridges, retainers, malocclusion and or clear aligners. These all complicate the removal of debris in certain areas of the oral cavity.

In accordance with an embodiment, tiny, active particles can be applied to teeth and gums and controlled to clean areas that toothbrush bristles and gloss cannot clean. The particles can be effective against common bacterial species that cause human oral diseases.

In accordance with a feature of the embodiments, a stringless oral debris removal system can be provided that includes a U-shaped mouth tray appropriately fitted to a dental ridge and teeth of a patient and having a trough defined by walls corresponding roughly to the height of the teeth and can extend 1 mm to 2 inches above or below the upper or lower gum line of the patient.

In accordance with another feature of the embodiments, the mouth tray can be optically clear thereby enabling penetration of visible, ultraviolet and infrared light, and the trough can be adapted to contain a ferrofluid comprising nanoscale or microscale magnetic particles applied to the teeth and gums of the patient.

In accordance with another feature of the embodiments, a magnet configured to emit a magnetic field to manipulate the ferrofluid mixture within the trough and around the teeth and gumline can be provided with the system.

In accordance with another feature of the embodiments, the mouth tray is made from a flexible material.

In accordance with another feature of the embodiments, the mouth tray can be formed in small, medium and large sizes and can be customized to fit patient upper and lower teeth and extend over the patient gum lines.

In accordance with another feature of the embodiments, a lighting device configured to emit light trough the U-shaped mouth tray and onto the ferrofluid, the teeth and the gums can be provided with the system.

In accordance with another feature of the embodiments, the lighting device can be provided in the form of light emitting diodes integrated within the U-shaped mouth tray.

In accordance with another feature of the embodiments, light emitted from the lighting device can be emitted in a combination of a combination of red (600-750 nm), blue (400-500 nm) and infrared (750-1100 nm) or white light (350-800 nm).

In accordance with another feature of the embodiments, the ferrofluid further can include a whitening ingredient such as carbamide peroxide and or hydrogen peroxide.

In accordance with another feature of the embodiments, the ferrofluid can include magnetic particles coated with a carbohydrate, molecules or polymers for colloidal stability and biofunctionalization.

In accordance with another feature of the embodiments, the ferrofluid can include a coating material including at least one of polyethylene glycol, dextran, alginate, or any hydrocarbon with a terminal carbonyl, carboxyl, or hydroxyl functional group or groups.

In accordance with another feature of the embodiments, the ferrofluid can contain hydroxyapatite nanoscale or microscale particles.

In accordance with another feature of the embodiments, the ferrofluid can contain carbomer.

In accordance with another feature of the embodiments, the ferrofluid can contain a preservative or a detergent such as sodium benzoate, paraben, sodium lauryl sulfate, cocamidopropyl betaine, alpha-tocopherol, or Cocos nucifera.

In accordance with another feature of the embodiments, the magnet can be comprised of iron, iron compounds, Molybdenum, NeFeB, or other magnetic materials.

In accordance with another feature of the embodiments, the magnet can be an electromagnet.

In accordance with another feature of the embodiments, the electromagnet can be configured to produce alternating magnetic fields.

In accordance with another feature of the embodiments, the magnet can include a handle for ergonomic use (e.g., insertion/removal) within the mouth.

In accordance with another feature of the embodiments, the magnetic can have a curved shape to easily connect with the u-shaped trough.

In accordance with another feature of the embodiments, the magnet can have a geometry configured for use with top and bottom mouth trays.

### BRIEF DESCRIPTION OF THE DRAWINGS

Specifics, details, generalized application, advantages, and summary of the present invention will be described visually via the following detailed description which in encompass optimized embodiments of this invention. The description references to the accompanying figures summarized below:

FIG. 1 is a schematic of the clear mouth tray showing the manipulation of the ferrofluid using an external static magnet attached to a handle for ergonomics.

FIG. 2 is a perspective view of the teeth and gums showing the accumulation of the magnetic particles between the teeth and at the gum line, targeting unreachable debris. These particles may be remotely controlled to scrub these areas, loosening adherent debris.

FIG. 3 is a lateral view of the patient's head as they use the device. Simulated magnetic field lines are shown.

FIG. 4 is a simulated view of the oral cavity showing untreated (yellow) teeth and treated (while clean teeth.

FIG. 5 is a perspective view of a mouth tray including lighting components therein, in accordance with an embodiment.

### DETAILED DESCRIPTION OF THE PREFERRED EMBODIMENTS

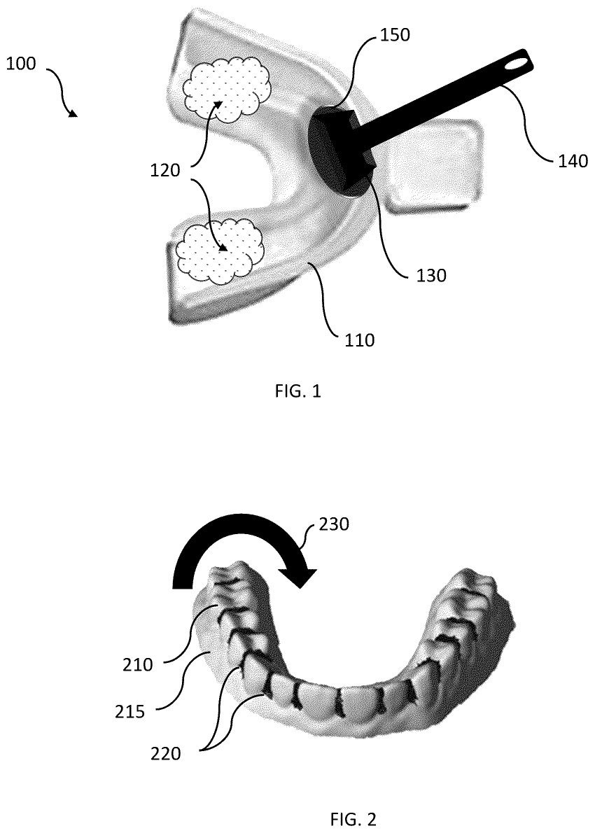

Referring to FIG. 1, illustrated is an embodiment of the invention 100 having three main components: 1.) a mouth tray 110 that can be optically transparent and flexible (e.g., clear aligner made of flexible polymeric material) with a basic U-shaped trough to fit over a patient's teeth and gum line (as seen in FIG. 1, this tray can be optimized to fit the dental ridges of the patient); 2.) a liquid or gel ferrofluid “floss” containing magnetic particles, herein referred to as ferrofluid floss 120, to be dispersed inside the tray 110; and 3.), and a magnet 130 that can include a handle 140 and a swivel head 150 for manipulation of the ferrofluid 120 within the clear tray 110.

Referring to FIG. 2, illustrated is a perspective view of the teeth 210 and gums 215 showing the accumulation of the magnetic particles 220 between the teeth and at the gum line, targeting unreachable debris. These particles may be remotely controlled to apply with magnetic influence 230 to the magnetic particles 220 to scrub areas between teeth and along gum lines, loosening adherent debris

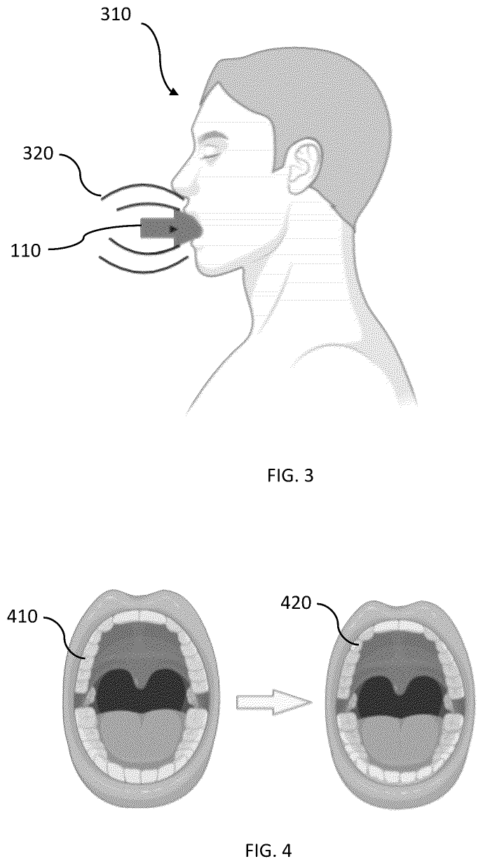

Referring to FIG. 3., illustrated is a lateral view of the patient's head as the patient uses the device described in FIG. 1. Simulated magnetic field lines 320 are shown when the tray 110 is placed over the patient's teeth and the ferrofluid floss 120 is applied inside the tray 110 (upper and lower trays), and the ferrofluid floss 120 is manipulated with the magnet 130 (static or electronic) attached to/in a handle 140. The user 310 can watch the particles move and scrub using a mirror. Prior to the application of the polymeric trays to the user, the ferrofluid floss 120 is applied to the insides of each tray 110.

The ferrofluid floss 120 treatment mixture can be comprised of nano or microparticles comprising iron, or an iron compound with ferri-, ferro, or superparamagnetic properties and coated with a polymer, carbohydrate, or other molecule in order to avoid agglomeration and to enhance solubility in the carrier fluid. A range of carrier fluids can be used, and they may be hydrophobic, hydrophilic or amphiphilic in nature. The fluid can also contain a whitening agent such as hydrogen peroxide, or in the optimized manifestation; carbamide peroxide. If hydrogen peroxide is used, the optimum concentration should be ≤10% and if carbamide peroxide is used, it can be at a concentration of ≤30%. In the optimized manifestation, the concentration of hydrogen peroxide would not exceed 5% and the concentration of carbamide peroxide would not exceed 15%. The mixture may also contain hydroxyapatite microscale (1 to 100 microns) or nanoscale (0.1 to 100 nm) particles for tooth remineralization. The mixture can also contain an antiseptic or antimicrobial agent or agents.

The optimized manifestation can encompass a mouth tray 110 comprised of a thin polymer, such as those used in clear aligners, as not to significantly shield the static magnetic field. The optimized manifestation can also include patient-specific mouth trays 110 to fit upper or lower teeth, in consideration of the architecture of the arch. The trays which were filled with ferrofluid floss 120 can be filled in such a volume as to cover the teeth and gums without spilling over into the mouth. The tray can fit in such a way that they allow movement of the particles but fit the teeth 210 and gums 215 snugly so as not to reduce mechanical action against adhered debris. The optimized manifestation can maximize ferrofluid contact with the teeth 210 and gums 215.

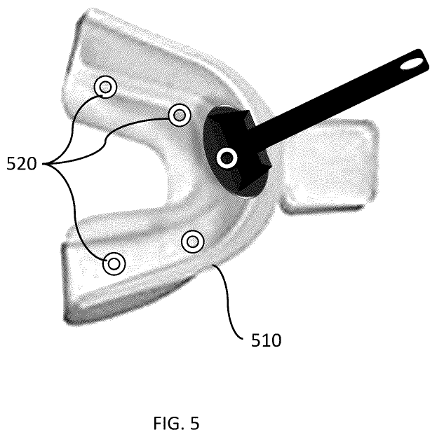

Referring to FIG. 5, illustrated is a mouth tray 510 like that illustrated in FIG. 1, but including lighting in accordance with an embodiment. For providing lighting, the mouth tray 510 can incorporate LEDs 520 in specific therapeutic wavelengths to encourage whitening (blue wavelengths) and or nanoparticle heating (red or infrared wavelengths). These LED's 520 can also be externally applied. Since the trays can be optically clear, the polymers can easily be engineered to contain color-tunable LEDs, or external LEDs that could be applied via an additional device. The shape of the mouth tray 510bis also expected to disperse the light waves, analogous to a convex-shaped lens when a light is used during treatment.

A magnet 130 when used to manipulate the particles can be attached to a handle 140 for ease of use and may be composed of any static magnetic material, such as NdFeB, Fe, Molybdenum, or other materials. In an alternative manifestation, the magnet can be comprised of an electromagnet. The electromagnet can emit alternating magnetic fields to cause manipulation of the ferrofluid mixture around the teeth and gum lines. The electromagnet 130 can also include a handle 140 for ease of use, or the electromagnet can also be incorporated into the mouth tray 110.

The present invention uses magnetic fields in a way of treating oral biofilms that is different than the methods that have been previously disclosed. The present invention utilizes a magnet 130 to activate movement of magnetic particles carried by ferrofluid fluid 120 in a U-shaped mouth trough of the tray 110 to control topical/orally applied magnetic nanoparticles in such a way that they become magnetized, as superparamagnetic nanoparticles are only magnetized in the presence of an external field. This is done so that the nanoparticles do not agglomerate prior to topical application. Ferromagnetic particles, would agglomerate in the tube and in the case of magnetic nanoparticle enhanced toothpaste, could not be colloidally dispersed in the paste or gel, and would not be distributed evenly upon application to the affected area. By activating the magnetic properties within the trough with a magnetic field, the nanoparticles can become magnetized after being applied to the affected area. A common complication of periodontitis is the onset of periodontal bone loss. EM fields can also be used to stimulate bone cell proliferation.

1. A stringless oral debris removal system, comprising:
a U-shaped mouth tray appropriately fitted to a dental ridge and teeth of a patient and having a trough defined by walls corresponding roughly to the height of the teeth and extending 1 mm to 2 inches above or below an upper or lower gum line of the patient, wherein the mouth tray is optically clear thereby enabling penetration of visible, ultraviolet and infrared light, and wherein the trough is adapted to contain a ferrofluid comprising nanoscale or microscale magnetic particles applied to the teeth and gums of the patient; and
a magnet configured to emit a magnetic field to manipulate the ferrofluid mixture within the trough and around the teeth and gumline.

2. The system of claim 1, wherein the mouth tray is made from a flexible material.

3. The system of claim 1, wherein the mouth tray is formed in small, medium and large sizes and is customizable to fit patient upper and lower teeth and extend over the patient gum lines.

4. The system of claim 1, further comprising a lighting device configured to emit light trough the U-shaped mouth tray and onto the ferrofluid, the teeth and the gums.

5. The system of claim 4, the lighting device further comprises light emitting diodes integrated within the U-shaped mouth tray.

6. The system of claim 4, wherein light emitted from the lighting device is emitted in a combination of a combination of red (600-750 nm), blue (400-500 nm) and infrared (750-1100 nm) or white light (350-800 nm).

7. The system claim 1, wherein the ferrofluid further comprises a whitening ingredient such as carbamide peroxide and or hydrogen peroxide.

8. The system of claim 1, wherein the ferrofluid comprises magnetic particles coated with a carbohydrate, molecule or polymer for colloidal stability and biofunctionalization.

9. The system of claim 1, wherein the ferrofluid comprises a coating material including at least one of polyethylene glycol, dextran, alginate, or any hydrocarbon with a terminal carbonyl, carboxyl, or hydroxyl functional group or groups.

10. The system of claim 1, wherein the ferrofluid contains hydroxyapatite nanoscale or microscale particles.

11. The system of claim 1, wherein the ferrofluid contains carbomer.

12. The system of claim 1, wherein the ferrofluid contains a preservative or a detergent such as sodium benzoate, paraben, sodium lauryl sulfate, cocamidopropyl betaine, alpha-tocopherol, or Cocos nucifera.

13. The system of claim 1, wherein the magnet is comprised of iron, iron compounds, Molybdenum, NeFeB, or other magnetic materials.

14. The system of claim 1, wherein the magnet is an electromagnet.

15. The system of claim 14, wherein the electromagnet is configured to produce alternating magnetic fields.

16. The system of claim 1, wherein the magnetic includes a handle for ergonomic use within the mouth.

17. The system of claim 1, wherein the magnetic has a curved shape to easily connect with the u-shaped trough.

18. The system of claim 1, wherein the magnetic has a geometry configured for use with top and bottom mouth trays.
